(19) United States
(12) Patent Application Publication
(10) Pub. No.: US 2025/0157598 A1
(43) Pub. Date: May 15, 2025

(54) COMPUTER-IMPLEMENTED METHOD FOR THE PROCESSING AND/OR CREATION OF CLINICAL TRIAL PROTOCOL DOCUMENTATION

(71) Applicant: Research Grid Ltd, London (GB)

(72) Inventors: Amber Michelle Hill, London (GB); Gautier Dagan, London (GB)

(21) Appl. No.: 18/505,696

(22) Filed: Nov. 9, 2023

(51) Int. Cl. G16H 10/20

(52) U.S. Cl. CPC G16H 10/20

(57) ABSTRACT

A computer-implemented method for the processing and/or creation of clinical trial protocol documentation including obtaining an indication of the registration body for the clinical trial; obtaining fields that need completing for each document that forms part of the clinical trial documentation; auto-populating some of the fields of the clinical trial documentation with suggested text; and outputting completed clinical trial documentation as a machine-readable file, in a format based on the indication of the registration body. The method may additionally comprise outputting “smart” documents for use in a trial master file.

### FIELD OF THE DISCLOSURE

The present disclosure relates to a computer-implemented method for the processing and/or creation of clinical trial protocol documentation.

### BACKGROUND

Trial protocols are documents that describe the objectives, design, methodology, statistical considerations, and aspects related to the organization of clinical trials. Trial protocols provide the background and rationale for conducting a study, highlighting specific research questions that are addressed, and taking into consideration ethical issues. Trial protocols must meet a standard that adheres to the principles of Good Clinical Practice and are used to obtain ethics approval by local Ethics Committees or Institutional Review Boards.

Throughout these documents there is repetitive text. There are also submitted several groups for approvals and each change in version of document is manually processed and costs a lot of time and money per document.

Furthermore, trial protocol documents need to be submitted to the relevant registry or registration body. However, many trial protocol documents are in an unstructured format. For example, there are thousands of long unstructured protocol textual data, that contains the definitions and explanations of each required answer in a protocol document. This unstructured data cannot be processed for guidance against the research information. To submit the trial protocol documents to the relevant registration body, and to enable changes to be translated and propagated across the many trial protocol documents, it would be advantageous if the data is in a machine-readable, structured, format.

Embodiments of the disclosure seek to address these problems.

### SUMMARY OF THE DISCLOSURE

Aspects of the invention are as set out in the independent claims and optional features are set out in the dependent claims. Aspects of the invention may be provided in conjunction with each other and features of one aspect may be applied to other aspects.

In a first aspect there is provided a computer-implemented method for the processing and/or creation of clinical trial protocol documentation. The method includes obtaining an indication of the registry or registration body for the clinical trial; obtaining fields that need completing for each document that forms part of the clinical trial documentation; auto-populating at least some of the fields of the clinical trial documentation with suggested text; and outputting completed clinical trial documentation as a machine-readable file, in a format based on the indication of the registration body. It will be understood that the machine-readable file may not actually be the clinical trial documentation itself, but when received by the relevant registration body, be suitable for easy conversion into clinical trial documentation.

The clinical trial documentation may be, or otherwise form part of, the trial master file (TMF). In some examples, the method may include outputting “smart” documents for use in the TMF. This may include using a machine-learning model to auto-populate regulatory documents and file them in a TMF with allocated metadata. The allocated metadata may provide a change log or automated change management—for example, the metadata may provide a date/time stamp, an indication of when the file was last modified and/or by whom etc. The machine-learning model may additionally or alternatively file documents in a filing structure in the TMF based on, for example, the header of the document, and/or headers of relevant sections of different fields of the document. The header itself may be determined by a machine-learning model based on the content of the file in question.

Advantageously, this enables clinical trial documentation to be prepared in an efficient and accurate manner. A user may be able to accurately enter relevant information relevant to their clinical trial, and clinical trial documentation may be prepared and output in a manner (e.g., in a structured data format) that can be easily imported by the clinical trial registration body.

In some examples, the method includes, determining which clinical trial documentation is required for that registration body based on the obtained indication of the registration body.

It will also be understood that in some examples the method may include amending or translating protocol documents originally created for one registration body to a format suitable for another registration body. Currently, a protocol document created for one registration body cannot be transferred/copied or migrated to another registration body. Embodiments of the disclosure enable protocol documents originally prepared in the format of a first registration body to be transformed and translated into a completely different format required by another registration body. This is due to the way in which the data may be structured as set out in more detail below.

As will be described in more detail below, embodiments of the disclosure allow relevant clinical trial protocol information (such as definitions) to be located and matched against keys for the required fields and then structured in a way that is presentable to a user but also suitable for automated processing to improve guidance against the research information.

This may be achieved by the combined use of multiple algorithms in a specific manner. For example, key detection may be used to analyze the data from internal and external regulatory documents to find the relevant definitions associated with each section of the document, which may then be processed using natural language processing (NLP) word tokenizer algorithms or Large Language Models (LLM) may be used to optimize the performance and the accuracy of the results, and to further segregate the data according to what output is needed. The new structured data may be presented to the user for example on a web interface for guidance and automated processing.

The obtained indication of the registration body may be obtained either by the user directly inputting the registration body (e.g., by a number associated with the registration body) and/or by the user adding or inputting their registration number which may indicate where the data is being parsed from. It may also indicate on the backend which of a plurality of respective libraries information is being pulled from. (e.g., suggestions, etc.).

In some examples, the machine-readable format may be an index. There may be two indexes, one for a Sponsor TMF and one for a Site TMF. An index gives a clear, standardized structure of the Sponsor and Site Files as well as guidance on what should be filed in each section.

Auto-populating at least some of the fields of the clinical trial documentation may include using a natural language processing (NLP) model or large language model (LLM) to determine section headers for the fields of the clinical trial documentation, and auto-populating at least some of the fields of the clinical trial documentation based on the corresponding determined section header. The section headers may include any of: Title Page, Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling, and Recordkeeping, Publication Policy, Project Timetable/Flowchart, References, and Supplements/Appendices.

Additionally, or alternatively, auto-populating at least some of the fields of the clinical trial documentation may include auto-populating at least some of the fields of the clinical trial documentation with suggested text based on the registration body and based on other documents uploaded and/or prepared by the user. Those other documents may be documents for the same clinical trial, or may be for other clinical trials. Other documents may be for other clinical trials for that same registration body or for other registration bodies.

Additionally, or alternatively, auto-populating at least some of the fields of the clinical trial documentation may include auto-populating at least some of the fields of the clinical trial documentation with suggested text based on other fields and/or documents completed by the user.

In another aspect, there is provided a computer-implemented method for the processing and/or creation of clinical trial protocol documentation. The method includes obtaining an indication of the registration body for the clinical trial; based on the obtained indication of the registration body, determining which clinical trial documentation is required for that registration body; creating template documents for completion by a user based on the determination of which clinical trial documentation is required for that registration body; suggesting text for inclusion in the template documents based on the registration body and based on text entered by the user in other documents or other sections of the same document; and outputting completed clinical trial documentation as a coded document, in a format based on the indication of the registration body. Advantageously this may enable clinical trial documentation, or information capable of creating clinical trial documentation, to be output in a standardised format to match that of the registration body.

Suggesting text for inclusion may include suggesting text based on the registration body and/or based on other documents uploaded and/or prepared by the user. Additionally, or alternatively, suggesting text for inclusion may include suggesting text based on other fields and/or documents completed by the user. Additionally, or alternatively, suggesting text for inclusion may include using a natural language processing model to determine section headers for the fields of the clinical trial documentation and providing a suggestion based on the relevant section header. The section headers may include any of: Title Page (General Information), Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling and Recordkeeping, Publication Policy, Project Timetable/Flowchart, References, and Supplements/Appendices.

In some examples, determining which clinical trial documentation is required for that registration body includes a lookup in a database of registered protocols for that registration body. The database may, for example, be a locally stored databased of registered protocols.

Outputting completed clinical trial documentation may include outputting clinical trial documentation in a format to match ICH GCP guidelines for TMF. Additionally, or alternatively, outputting completed clinical trial documentation also includes outputting meta data with the clinical trial documentation, wherein the meta data includes at least one of: time stamp, history log, version control. Additionally, or alternatively, outputting completed clinical trial documentation also includes outputting a human-readable set of clinical trial documentation.

In some examples, the output completed clinical trial documentation are restricted from editing.

The method of either aspect may include obtaining other documents uploaded and/or prepared by the user and converting these to a first machine-readable format, and wherein outputting completed clinical trial documentation includes converting the completed clinical trial documentation to a second format, wherein the second format is based on the indication of the registration body. It will be understood that in some examples, documents may be converted from the second format (for a first registration body) to another (third) format (for example, for a second registration body).

The method of either aspect may include obtaining other documents for that registration body, and converting these to a first machine-readable format, and wherein outputting completed clinical trial documentation as a coded document, includes converting the completed clinical trial documentation to a second format, wherein the second format is based on the indication of the registration body. The obtained other documents for that registration body may be part of a library of documents for that registration body.

In some examples, subsequent to outputting completed clinical trial documentation, the method include: receiving a request by a user to make a change to data in a first document; and implementing the requested change to all other documents where that same data is used in other documents for that same clinical trial.

In another aspect there is provided a computer-implemented method for the processing and/or creation of clinical trial protocol documentation. The method includes obtaining completed clinical trial documentation; receiving a request by a user to make a change to data in a first document; and implementing the requested change to all other documents for the same clinical trial where that same data is used.

In another aspect there is provided a computing device including a display screen, the computing device being configured to display on the screen: a prompt to a user to provide an indication of a clinical trial registration body; a prompt to a user to enter in relevant clinical trial information; a prompt to a user to accept suggested content for fields of the clinical trial documentation; and a prompt to the user to generate and export clinical trial documentation in a format based on the clinical trial registration body.

In another aspect there is provided a computer readable non-transitory storage medium including a program for a computer configured to cause a processor to perform the method of any of the aspects described above.

### BRIEF DESCRIPTION OF THE DRAWINGS

Embodiments of the disclosure will now be described, by way of example only, with reference to the accompanying drawings, in which:

FIG. 1 shows a functional schematic overview of an example system for processing and/or creating clinical trial protocol documentation.

FIG. 2 shows a functional schematic overview of an example system for processing and/or creating clinical trial protocol documentation.

FIG. 3 shows the example system of FIG. 2 but with the addition of a user selection step and a remote data store.

FIG. 4 shows a screenshot of an initial page of a graphical user interface a user may be presented with when wishing to create and/or process clinical trial protocol documentation.

FIG. 5 shows a screenshot of a subsequent page of a graphical user interface the user may then be presented with.

FIG. 6 shows a screenshot of a subsequent page of a graphical user interface the user may then be presented with.

FIG. 7 shows a screenshot of a subsequent page of a graphical user interface the user may then be presented with.

FIG. 8 shows a screenshot of a subsequent page 900 the user may then be presented with.

FIG. 9 shows a screenshot of another page of a graphical user interface the user may be presented with.

FIG. 10 shows a screenshot of another page of a graphical user interface the user may be presented with.

FIG. 11 shows a screenshot of another page of a graphical user interface the user may be presented with.

FIG. 12 shows how the system may be used to output clinical trial documentation in a structured format, which in this case is xml format.

FIG. 13 shows how the system may be used to output clinical trial documentation in a human-readable format, which in this case is pdf format.

FIG. 14 shows a screenshot of a graphical user interface showing how the system may auto-generate or auto-populate fields based on uploaded clinical trial documentation.

FIG. 15 shows a process flow chart of an example computer-implemented method for the processing and/or creation of clinical trial protocol documentation.

FIG. 16 shows a process flow chart of another example computer-implemented method for the processing and/or creation of clinical trial protocol documentation.

FIG. 17 shows a process flow chart of another example computer-implemented method for the processing and/or creation of clinical trial protocol documentation.

FIG. 18 shows a process flow chart of how the graphical user interface may interact with a user to enable operation of the example computer-implemented methods described above.

FIG. 19 shows a process flow chart of another example computer-implemented method for the processing and/or creation of clinical trial protocol documentation.

FIG. 20 shows an exemplary device configured to enable and/or perform some or all of the functionality discussed herein.

Like reference numbers denote features with the same or similar functionality.

### DETAILED DESCRIPTION

Every clinical trial has a series of documents spanning hundreds of pages outlining the methodology of the trial, its infrastructure, emergency procedures, lay recruitment information, and more. Throughout these documents there is repetitive text. There are also submitted several groups for approvals and each change in version of document is manually processed and costs a lot of time and money per document.

The International Council for Harmonisation of Technical Requirements for Pharmaceuticals for Human Use (ICH) Guideline for Good Clinical Practice (GCP) sets out that Trial Master Files (TMFs) should be established at the beginning of the trial, both at the investigator/institution's site and at the sponsor's office. A final close-out of a trial can only be done when the monitor has reviewed both investigator/institution and sponsor files and confirmed that all necessary documents are in the appropriate files.

The TMF can be defined as the collection of essential documents that is used by Sponsors and Investigators/Sites for the management of a study and by the Monitor, Auditors and Inspectors to review and verify whether the Sponsor and the Investigator/Site have conducted the study in line with the applicable ethics and regulatory requirements and the principles and standards of GCP.

A minimum list of essential documents is mandated in Section 8 of the ICH GCP guideline (Note for Guidance on Good Clinical Practice, CPMP/ICH/135/95 July 2002) but it is always expected that a TMF will contain more than just this list, as appropriate to the study. The TMF is composed of a Sponsor TMF (sponsor file), held by the sponsor organisation, and a Site TMF (Site File) held by the Investigator/Site. The set up and maintenance of the TMF is the whole study team's responsibility.

It is good practice for a TMF to follow a standardised index. There may be two indexes, one for Sponsor Files and for Site Files. The indexes give a clear, standardized structure of the Sponsor and Site Files as well as guidance on what should be filed in each section.

These forms are reviewed by the study team when the TMF is set up and any sections that are not applicable to the study can be removed so that the Sponsor and/or Site File index/es are tailored to the study.

A tailored TMF means less need for multiple File Notes to explain empty Sponsor/Site File sections that are not applicable to a study.

A TMF may follow a folder structure—with sub folders for each section e.g.,
1. Site File Structure and QC
2. Trial Specific Documentation
3. Site Documentation
4. Evidence of Ethics
5. Safety
6. Governance
7. Data Management
8. Monitoring
9. Pharmacovigilance
10. Meetings
11. Laboratory
12. Device
13. Covid 19 pandemic

According to the ICH Good Clinical Practice guidelines, a protocol should include the following topics:
Title Page (General Information)
Background Information
Objectives/Purpose
Study Design
Selection and Exclusion of Subjects
Treatment of Subjects
Assessment of Efficacy
Assessment of Safety
Adverse Events
Discontinuation of the Study
Statistics
Quality Control and Assurance
Ethics
Data handling and Recordkeeping
Publication Policy
Project Timetable/Flowchart
References
Supplements/Appendices

The TMF and requirements for the TMF, as well as the clinical trial documentation itself, may vary based on the clinical trial regulation body or authority.

It can therefore be seen how preparing documentation for the TMF may be time consuming, burdensome and prone to error. There may be many sections of repetitive text and conflict may arise due to human errors introduced in preparing the documentation. Furthermore, the exact requirements for each TMF may vary based on the clinical trial registry or registration body, and it can be difficult and laborious to create protocol documentation for different registration bodies.

Accordingly, embodiments of the disclosure relate to a computer implemented method for the processing and/or creation of clinical trial documentation. The clinical trial documentation may be, contribute or otherwise form part of the TMF. In particular, embodiments of the claims provide a standardised and straightforward way for preparing clinical trial documentation that can then be uploaded to a clinical trial registry in a format specific to that registry. This means that a user can enter any relevant information in a standardised way, and that information converted and output in a format suitable for the relevant registration authority. Furthermore, embodiments of the disclosure enable elements of the process to be automated and for intelligent suggestions to be provided to the user as they are entering in information for the clinical trial documentation. This may greatly improve the speed, accuracy, and efficiency of the creation of clinical trial protocol documentation.

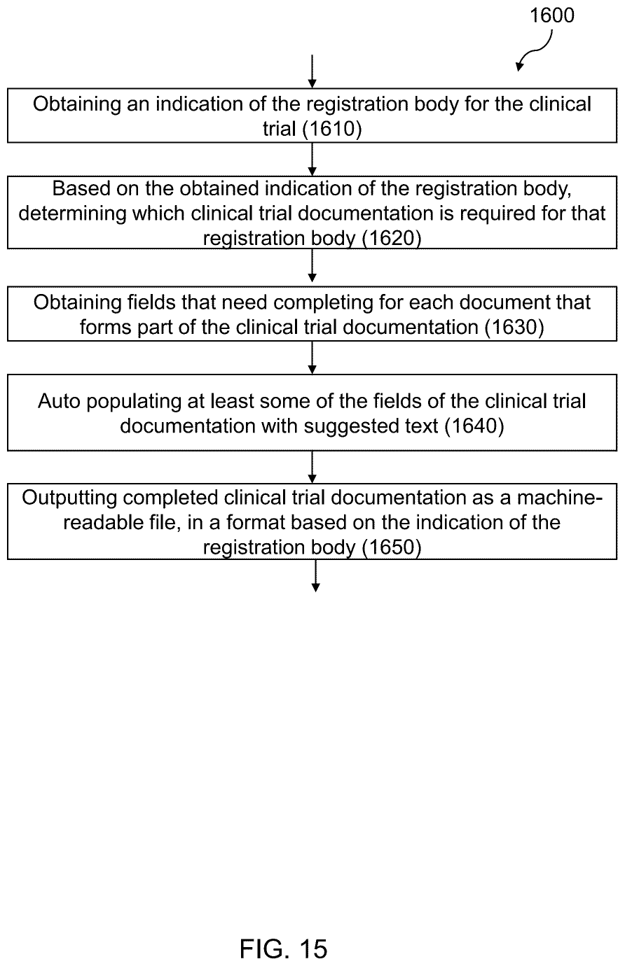

The prepared clinical trial documentation may be, or otherwise form part of, the trial master file (TMF). Embodiments of the disclosure may therefore include outputting “smart” documents for use in the TMF (where the “smart” documents may include, for example, auto-populated documents prepared for that regulatory body). This may include using machine learning to auto-populate regulatory documents and file them in a TMF with allocated metadata. The allocated metadata may provide a change log or automated change management—for example, the metadata may provide a date/time stamp, an indication of when the file was last modified and/or by whom etc. Embodiments of the disclosure may additionally or alternatively include filing documents in a filing structure in the TMF. The filing of documents and the filing structure may be based on, for example, the header of the document and/or the relevant regulatory body, and/or headers of relevant sections of different fields of the document as discussed below for example with reference to FIG. 15. In some examples, the header itself may be determined by a machine-learning model based on the content of the file in question.

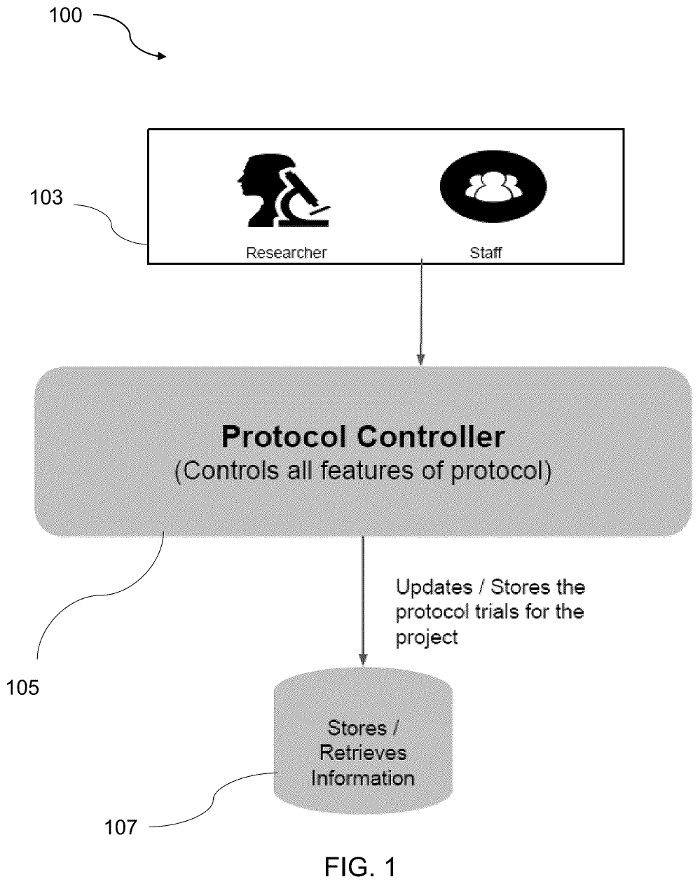

FIG. 1 shows a functional schematic overview of an example system 100 for processing and/or creating clinical trial protocol documentation. FIG. 1 shows the relationship between a user 103 (whether that is a researcher, or a staff member involved in the clinical trial), the protocol controller 105 (which will be described in more detail below) and the data store 107. The protocol controller 105 may also be called the anonymization controller as it may be configured to anonymize specific information such as the identity of the principal investigator. The protocol controller 105 may be configured to process and/or create clinical trial documentation. The protocol controller 105 is configured to control the parameters for the unstructured import, to the structured maintenance within the system, and then the structured export.

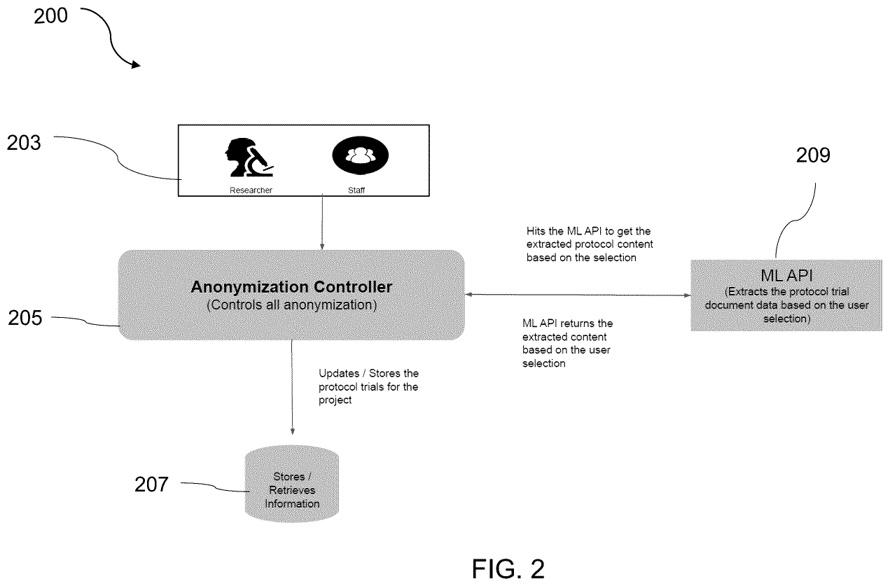

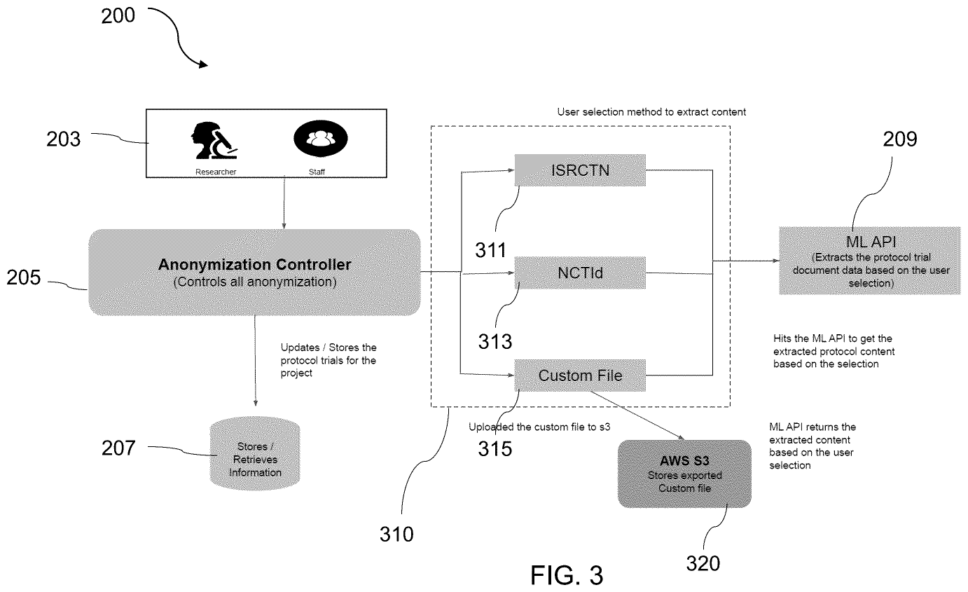

FIG. 2 shows a functional schematic overview of an example system 200 for processing and/or creating clinical trial protocol documentation. FIG. 2 shows the relationship between a user 203 (whether that is a researcher, or a staff member involved in the clinical trial), the anonymization controller 205 (which will be described in more detail below) and the data store 207. As can be seen in FIG. 2 the anonymization controller 205 is operable to receive information from a user 203 and communicate with the data store 207 to store and/or retrieve information. The anonymization controller 205 is also configured to communicate with a machine learning application programming interface (ML API) 209. The ML API 209 is configured to extract protocol trial document data based on the user selection. The user selection is shown in more detail in FIG. 3.

The protocol controller 105 controls the basic import, maintenance, and export of the document, whereas the anonymization controller 205 is a component of the protocol controller 105, that is optional for the user, which solely controls the anonymization of personal identifiable information within the document passed at the import.

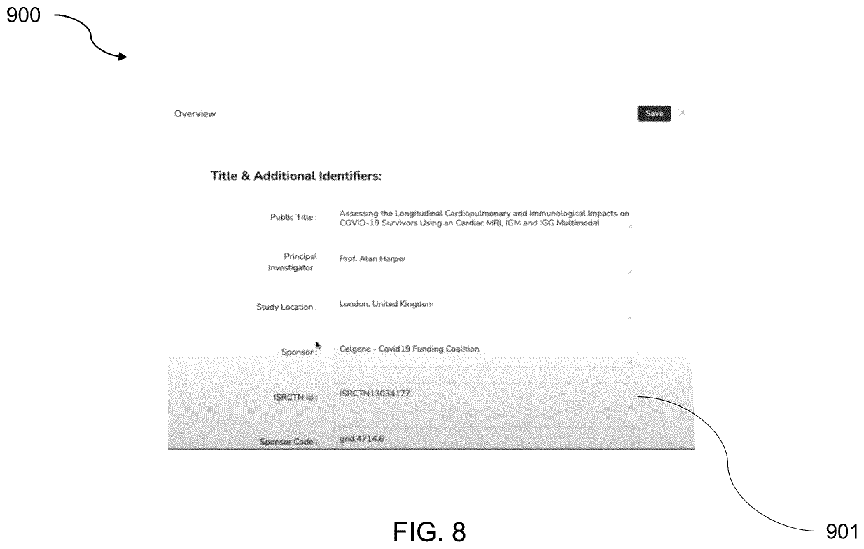

FIG. 3 shows the example system 200 of FIG. 2 but with the addition of a user selection step 310 and a remote data store 320. The user selection step 310 may involve the selection of a plurality of different clinical trial protocol registration bodies, such as ISRCTN 311 or NCTId 313 (these are of course only two examples and there are many other registration bodies including for example EUDRACT as shown in FIG. 8). The system may be pre-configured to prepare clinical trial protocol documentation for registration bodies that are listed and selectable by a user. However, the user may also be able to select a “Custom File” 315 category, which enables a user to import their custom protocol. This custom protocol may be uploaded to and accessed via a remote store 320 such as AWS S3. The call made to the ML API 209 is then based on the user selection 310 of which registered protocol they are using.

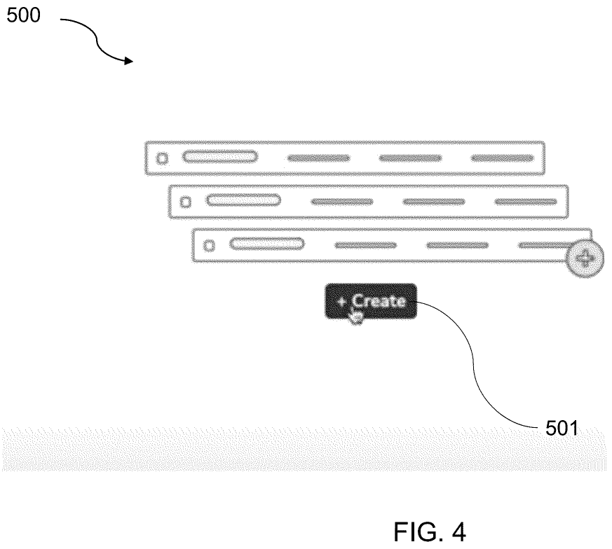

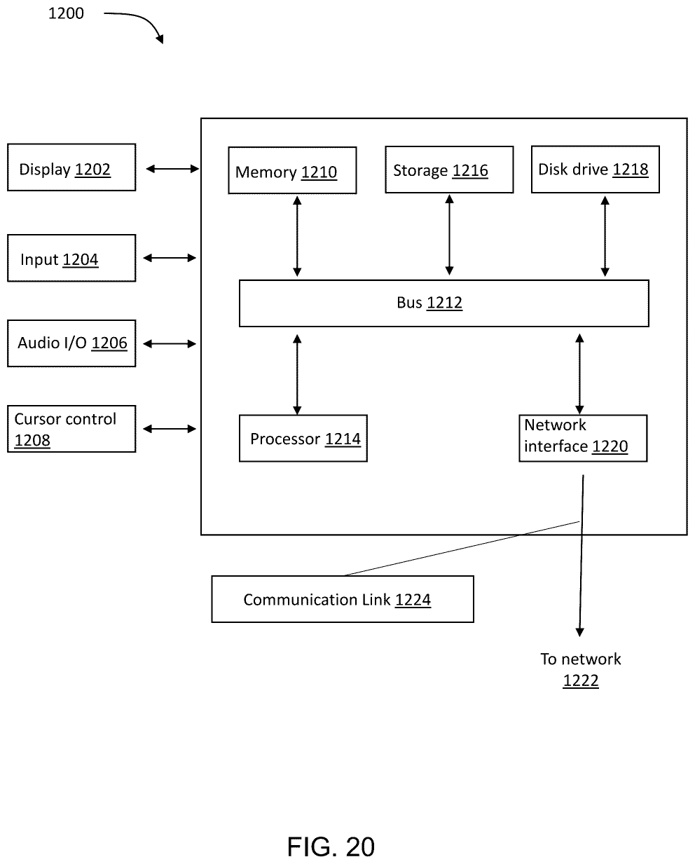

FIGS. 4 to 11 and 14 show example screenshots of an example graphical user interface of a system for processing and/or creating clinical trial protocol documentation. The graphical user interface may be operating on a computing device including a display screen, such as the example computer system described below with reference to FIG. 20.

FIG. 4 shows a screenshot of an initial page 500 a user may be presented with when wishing to create and/or process clinical trial protocol documentation. The initial page 500 may include a button 501 labelled “Create” that a user can click on to begin the process.

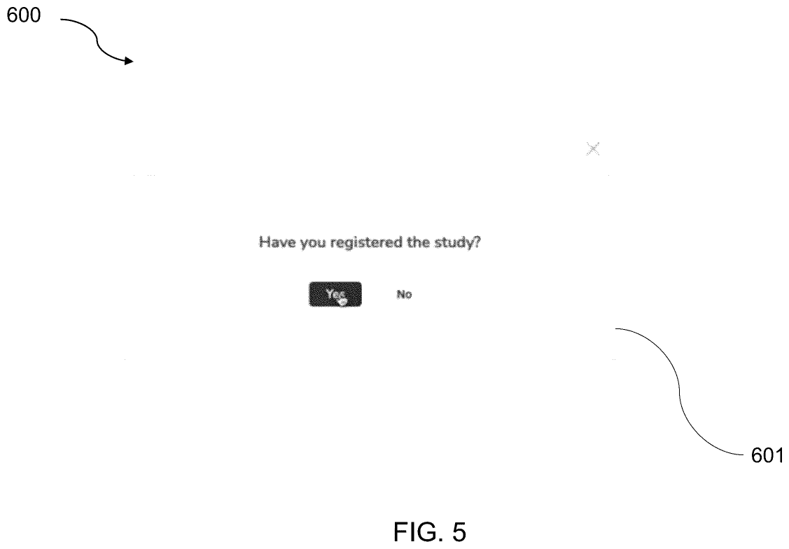

FIG. 5 shows a screenshot of a subsequent page 600 the user may then be presented with. This page asks the user via a caption box 601 whether the study has been registered, and presented the user with a binary option of selecting “yes” or “no”.

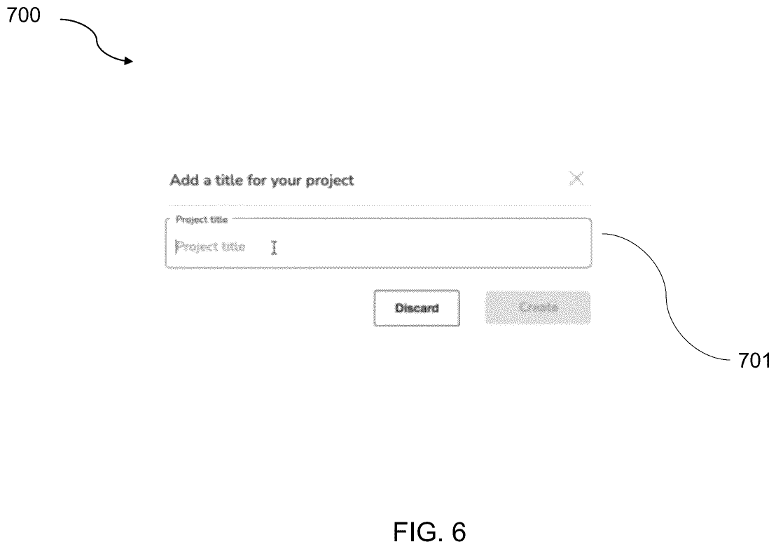

FIG. 6 shows a screenshot of a subsequent page 700 the user may then be presented with. This page asks the user via a caption box 701 to enter a title for the project.

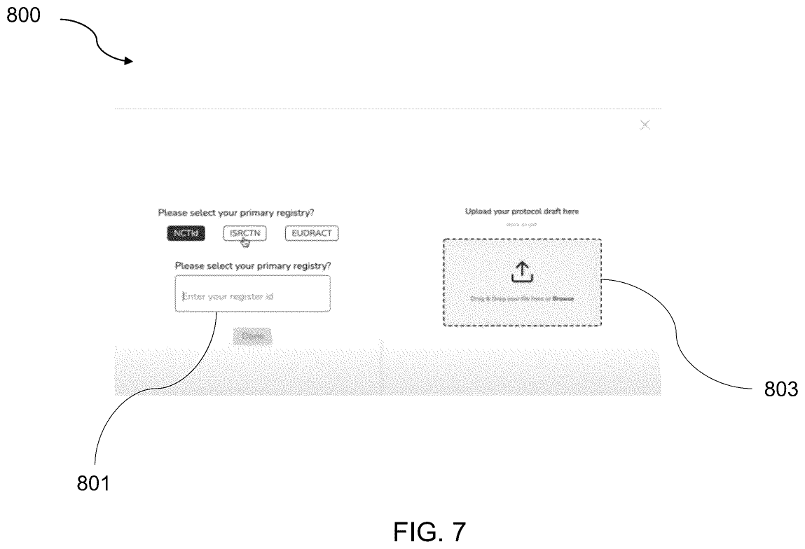

FIG. 7 shows a screenshot of a subsequent page 800 the user may then be presented with. This page asks the user to select which registry (i.e., registration body) is their primary registry and to enter the register ID via caption box 801. In some examples, the registry body may also be obtained from the user's ID. It is of course possible, as noted above with reference to FIG. 3, for the user to enter in a different or “custom” registry, which may involve the user uploading pre-filled or template documentation for that registry if it is not pre-stored by the system. If there's a registry not present and “custom registry” is selected, a user can still upload protocol documents in the same manner, but they may be structured by the system in a different way. The same steps occur from the protocol controller 105.

FIG. 8 shows a screenshot of a subsequent page 900 the user may then be presented with. This page presents much of the relevant information required by the system from a user to create clinical trial protocol documentation in the form of text boxes 901 the user can select or enter text into. The information may be grouped under “headers”, for example as set out below:

### Title & Additional Identifiers

public title
principal investigator
study location
Sponsor
ISRCTN Id
Sponsor code
Principal Investigator address
Trial coordinator
Sponsor representative
Co-investigators
Collaborators
Statistician
Central Laboratories

### Study Information:

Full title of study
Short tile of study
clinical phase
disease type
study design
primary objective
secondary objective
inclusion criteria
exclusion criteria
investigation medicinal product
participant group
total number of participants
study hypothesis
ethics approval
trial setting
trial type
condition
intervention type
primary outcome measure
secondary outcome measure

The user may then be able to click “save” to store this information, for example on data store 107, 207 as shown in FIGS. 1 to 3.

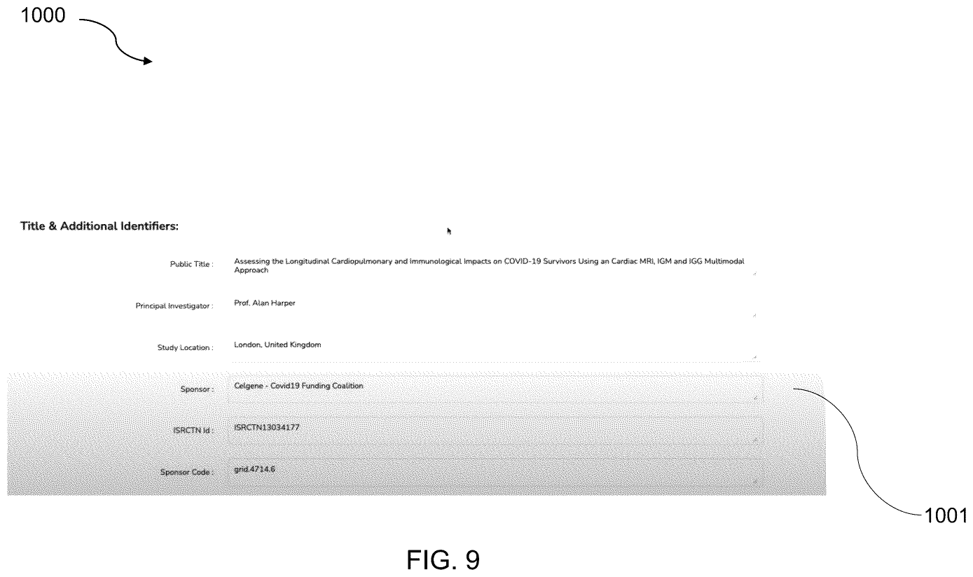

FIG. 9 shows a screenshot of another page 1000 the user may be presented with. Page 1000 is very similar to page 900 except it includes the following buttons the user may be able to click on: “change primary registry”, “generate registry upload document”, “generate protocol document”, and “generate study document”.

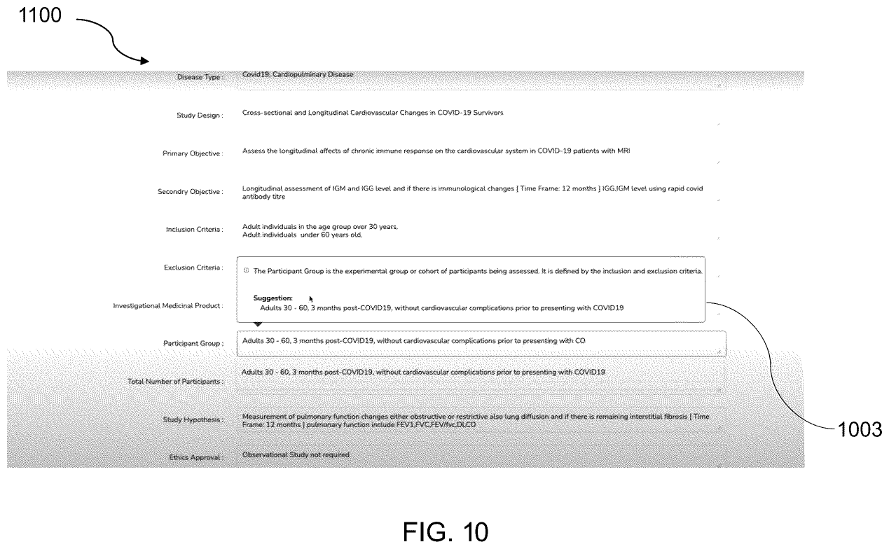

FIG. 10 shows a screenshot of another page 1100 the user may be presented with. Page 1100 is very similar to pages 900 and 1000. Page 1100 shows how the system may present the user with suggested text. In this example, when the user has selected a text box for entry, such as the box “participant group”, there is a bubble 1003 that appears above this text box. In the present case the bubble 1003 provides a summary of what is meant by the field in question—in this case, the summary explains what “Participant Group” means—it says that “The Participant Group is the experimental group or cohort of participants being assessed. It is defined by the inclusion and exclusion criteria.” Beneath the explanation of the relevant field, there is a “suggestion” for suggested text. In the present case, the suggested text suggests: “Adults 30-60, 3 months post-COVID19, without cardiovascular complications prior to presenting with COVID19.” The suggested text may be determined, for example, by the ML API 209 described above with reference to FIGS. 2 and 3. The suggested text may be suggested based on text entered by the user in other documents or other sections of the same document. For example, in the present case the suggested text may be based, at least, on text entered into the preceding “inclusion criteria” and “exclusion criteria” fields.

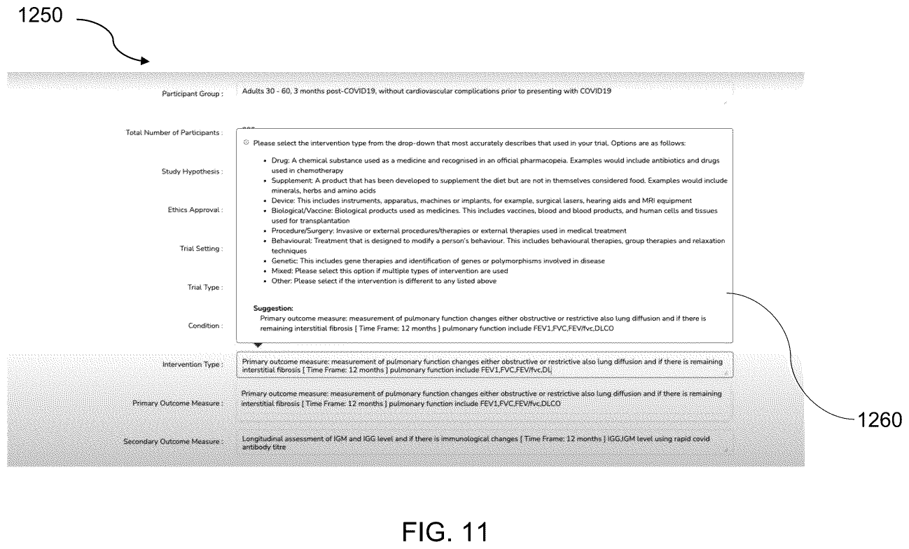

FIG. 11 shows a screenshot of another page 1250 the user may be presented with. Page 1250 is very similar to pages 900, 1000 and 1100. Page 1250 shows how the system may present the user with suggested text. In this example, when the user has selected a text box for entry, such as the box “Intervention Type”, there is a bubble 1260 that appears above this text box. In the present case the bubble 1260 explains to the user that they can select from a select range of options what the intervention type is. Below this explanation, there is a suggestion to the user of what the intervention type could be. The suggested text may be determined, for example, by the ML API 209 described above with reference to FIGS. 2 and 3. As with the example of FIG. 10, the suggested text may be suggested based on text entered by the user in other documents or other sections of the same document.

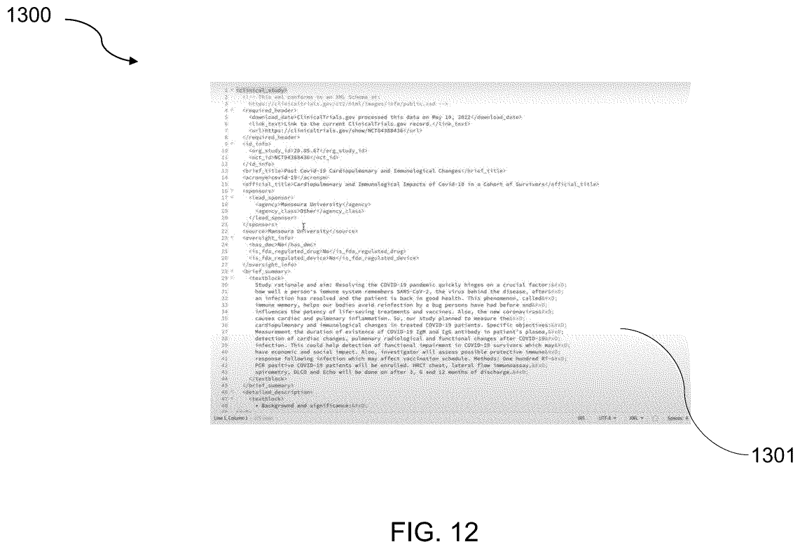

FIG. 12 shows how the system may be used to output clinical trial documentation in a structured format, which in this case is xml format. In the example shown, FIG. 12 shows a page 1300 displaying xml text 1301. The specific format in which the clinical trial documentation is output may be based on the clinical trial registration body/authority selected by the user, as the system may be configured to convert the data entered via the user interface into a machine-readable format that is readily received and interpreted by that relevant clinical trial registration body/authority.

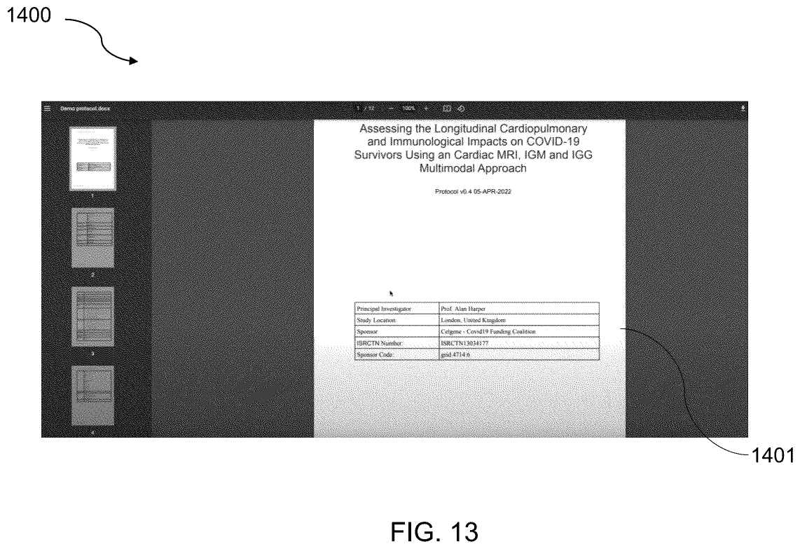

FIG. 13 shows how the system may be used to output clinical trial documentation in a human-readable format, which in this case is pdf format. In the example shown, FIG. 13 shows a page 1400 displaying xml text 1401. It will be understood that the system may be configured to output clinical trial documentation in both a human readable format (e.g., via a web interface) and a machine-readable format. In both instances, the output documentation may be formatted or structured in a way suitable for the relevant clinical trial registration body/format. For example, the documents may be structured in a particular way (e.g., have particular headers and fields) unique to that registration body/authority.

For example, the user inputs their protocol registration number into the system and it automatically acquires the unstructured data from the relevant public registry. The data is then parsed, and algorithms automatically execute a key value pairing that converts the unstructured data into structured data. A key-value pair consists of two related data elements: A key, which is a constant that defines the data set and a value, which is a variable that belongs to the set. Executing a key value pairing may include identifying the keys and pairs and matching them to each other. This may include, for example, using an OCR model (such as Tesseract OCR) to sequence inputs, and an image processing library. For example, the OpenCV library may be used for image reading and processing, and PyTesseract library may be used for OCR. Another machine learning model may (such as a long short term memory, LSTM, model) then be used to take the recognised text as inputs and outputs of the key value pairs.

The user may then send a request to the system to recreate their data for the public registry. This triggers the algorithm to use the newly structured data to render the desired (restructured) output for the selected public registry. The outputs are either a read-friendly pdf file, or a machine-readable file that can be used to transfer data to the other registry. It will be understood that this request may be repeated, so that newly structured data may be created for more than one registry.

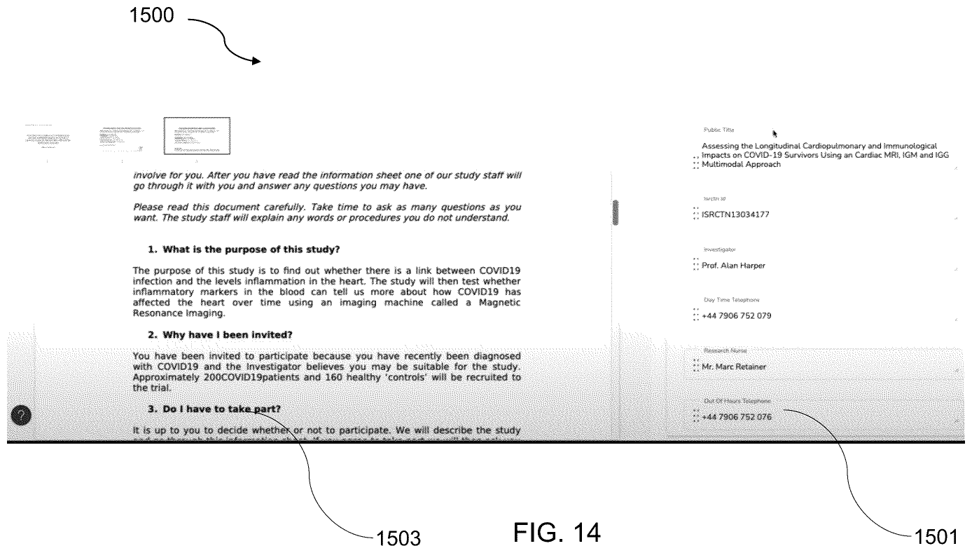

FIG. 14 shows how the system (for example, the ML API 209 of FIGS. 2 and 3) may auto-generate or auto-populate fields 1501 based on uploaded clinical trial documentation 1503.

FIG. 15 shows a process flow chart of an example computer-implemented method 1600 for the processing and/or creation of clinical trial protocol documentation. The method 1600 begins at step 1610 with obtaining an indication of the registration body for the clinical trial (for example, as described above with reference to FIG. 8). Based on the obtained indication of the registration body, the method includes the step of determining 1620 which clinical trial documentation is required for that registration body. The method then includes obtaining 1630 fields that need completing for each document that forms part of the clinical trial documentation. The fields that need completing may vary based on the clinical registration body and/or the documents required for that registration body—some fields may be required for select registration bodies and not others, for example. At least some of the fields of the clinical trial documentation may then be auto-populated 1640 with suggested text, as described above for example with reference to FIGS. 10 and 11. The suggested text may be based on the indication of the registration body, other clinical trial documentation completed and/or uploaded, and/or other fields already completed and/or uploaded. The suggested text may also be based on the relevant headers of the corresponding fields. Completed clinical trial documentation is then output 1650 as a machine-readable file (for example, an xml file). The format of the output clinical trial documentation may be based on the indication of the registration body. In some examples, both a machine-readable (e.g., xml) file and human-readable (e.g., pdf) file may be output.

It will be understood that the output clinical trial documentation may be in the form of an index or may be configured to provide an index to form the TMF, for example the Sponsor TMF and Site TMF. An index gives a clear, standardized structure of the Sponsor and Site Files.

The step of auto-populating 1640 at least some of the fields of the clinical trial documentation may include using a natural language processing model (for example, provided by the ML API 209 described above with reference to FIGS. 2 and 3). The natural language processing model may, for example, be configured to determine section headers for the fields of the clinical trial documentation, and auto-populate at least some of the fields of the clinical trial documentation based on the corresponding determined section header. The section headers may include any of: Title Page, Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling and Recordkeeping, Publication Policy, Project Timetable/Flowchart, References, and Supplements/Appendices.

As described above with reference to FIGS. 10 and 11, auto-populating at least some of the fields of the clinical trial documentation may include auto-populating at least some of the fields of the clinical trial documentation with suggested text based on the registration body and based on other documents uploaded and/or prepared by the user. The other documents may be for the same clinical trial or may be for other clinical trials. Other documents may be for other clinical trials for that same registration body or for other registration bodies.

As described above with reference to FIGS. 10 and 11, auto-populating at least some of the fields of the clinical trial documentation includes auto-populating at least some of the fields of the clinical trial documentation with suggested text based on other fields and/or documents completed by the user.

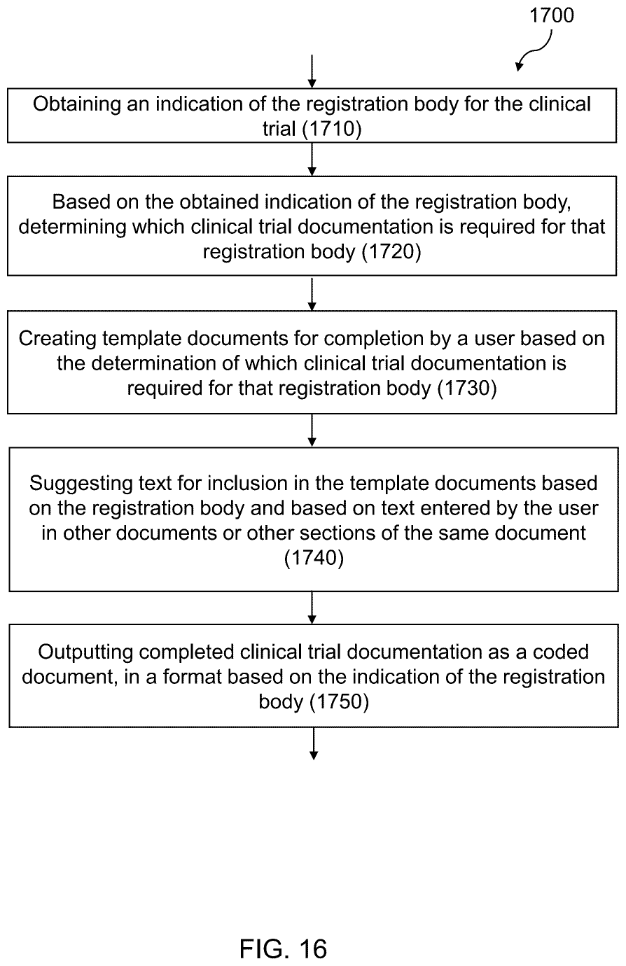

FIG. 16 shows a process flow chart of another example computer-implemented method 1700 for the processing and/or creation of clinical trial protocol documentation. The method includes the steps of obtaining 1710 an indication of the registration body for the clinical trial (for example, as described above with reference to FIG. 7), and then based on the obtained indication of the registration body, determining 1720 which clinical trial documentation is required for that registration body. Template documents for completion by a user are then created 1730 based on the determination of which clinical trial documentation is required for that registration body. Text for inclusion in the template documents is suggested 1740 (for example, as described above with reference to FIGS. 10 and 11) based on the registration body and based on text entered by the user in other documents or other sections of the same document. Completed clinical trial documentation is then output 1750 as a coded document, in a format based on the indication of the registration body. A coded document may be a machine-readable document, for example in xml, json or csv format.

Suggesting text for inclusion may include suggesting text based on the registration body and/or based on other documents uploaded and/or prepared by the user. Suggesting text for inclusion may include suggesting text based on other fields and/or documents completed by the user.

Suggesting text for inclusion may include using a natural language processing model (for example, provided by the ML API 209 described above with reference to FIGS. 2 and 3). The natural language processing model may, for example, be configured to determine section headers for the fields of the clinical trial documentation, and auto-populate at least some of the fields of the clinical trial documentation based on the corresponding determined section header. The section headers may include any of: Title Page, Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling and Recordkeeping, Publication Policy, Project Timetable/Flowchart, References, and Supplements/Appendices.

Determining 1620, 1720 which clinical trial documentation is required for that registration body may include a lookup in a database of registered protocols for that registration body. For example, registration bodies may have a library or libraries of registered protocols, and the system may be configured to create a local copy of these.

Outputting 1650, 1750 completed clinical trial documentation may include outputting clinical trial documentation in a format to match ICH GCP guidelines for TMF.

Outputting 1650, 1750 completed clinical trial documentation may additionally or alternatively also include outputting meta data with the clinical trial documentation, wherein the meta data includes at least one of: time stamp, history log, version control.

Outputting 1650, 1750 completed clinical trial documentation may also include outputting a human-readable set of clinical trial documentation, as described above with reference to FIG. 13. The output completed clinical trial documentation may be restricted from editing.

In some examples, the method of FIG. 15 or FIG. 16 may also include the step of obtaining other documents uploaded and/or prepared by the user and converting these to a first machine-readable format, and wherein outputting 1650, 1750 completed clinical trial documentation includes converting the completed clinical trial documentation to a second format. The second format may be based on the indication of the registration body.

In some examples, the method of FIG. 15 or FIG. 16 may also include obtaining other documents for that registration body, and converting these to a first machine-readable format, and wherein outputting completed clinical trial documentation as a coded document, includes converting the completed clinical trial documentation to a second format, wherein the second format is based on the indication of the registration body. The other documents for that registration body may be part of a library of documents for that registration body.

In some examples, subsequent to outputting 1650, 1750 completed clinical trial documentation, the method includes: receiving a request by a user to make a change to data in a first document; and implementing the requested change to all other documents where that same data is used in other documents for that same clinical trial.

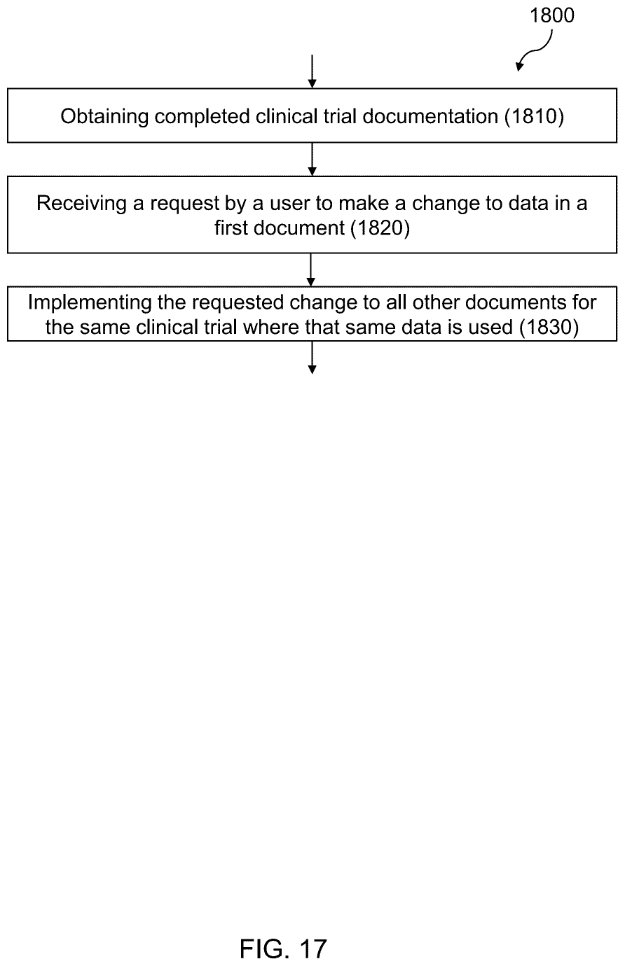

It will be understood that after clinical trial documentation has been created, that edits, changes or updated may need to be made to the documentation. Due to large number of documents involved, this can be a very laborious task significantly prone to error. Embodiments of the disclosure provide an efficient and accurate way to perform this, as will be described with reference to FIG. 17 in more detail below.

FIG. 17 shows a process flow chart of another example computer-implemented method 1800 for the processing and/or creation of clinical trial protocol documentation. The method 1800 includes initially obtaining 1810 completed clinical trial documentation. A request by a user to make a change to data in a first document may then be received 1810, and the requested change implemented 1830 to all other documents for the same clinical trial where that same data is used. The requested change may a change of information, such as text or numerical values, whether that be in the form of correction, addition or removal of that information.

The request made by the user may be a request to add or amend a relevant field, for example to correct or add information to a field, for example as entered according to the process described above with reference to FIGS. 8 to 11. Accordingly, the method may further include the step of identifying all instances in all the clinical trial documentation of the relevant field, or text/information based on that entered in the relevant field, and implementing 1830 the requested change may include replacing or amending all instances of that field, or text/information entered into that field, throughout the clinical trial documentation. It will be understood that identifying all instances in all the clinical trial documentation of the relevant field, or text/information based on that entered in the relevant field, may include the use of a machine learning model, for example a natural language processing (NLP) machine learning model.

Additionally, or alternatively, the request made by the user may be a request to add or amend a section. The new section may include a new section header. Accordingly, the method may further include the step of identifying all instances in all the clinical trial documentation that reference this field, or that might need to reference this section, and implementing 1830 the requested change may include adding the information contained in the new section to any clinical trial documentation that needs to reference this new information.

The request made by the user may be a request to remove a relevant field, or text within a field. Accordingly, the method may further include the step of identifying all instances in all the clinical trial documentation of the relevant field, or text/information based on that entered in the relevant field, and implementing 1830 the requested change may include replacing or amending all instances of that field, or text/information entered into that field, throughout the clinical trial documentation.

Implementing 1830 the requested change may further include updating meta data and/or a header or footer of any amended clinical trial documentation. This may include updating the clinical trial documentation with information identifying the user requesting the change or addition, the date and/or time of the requested change or addition.

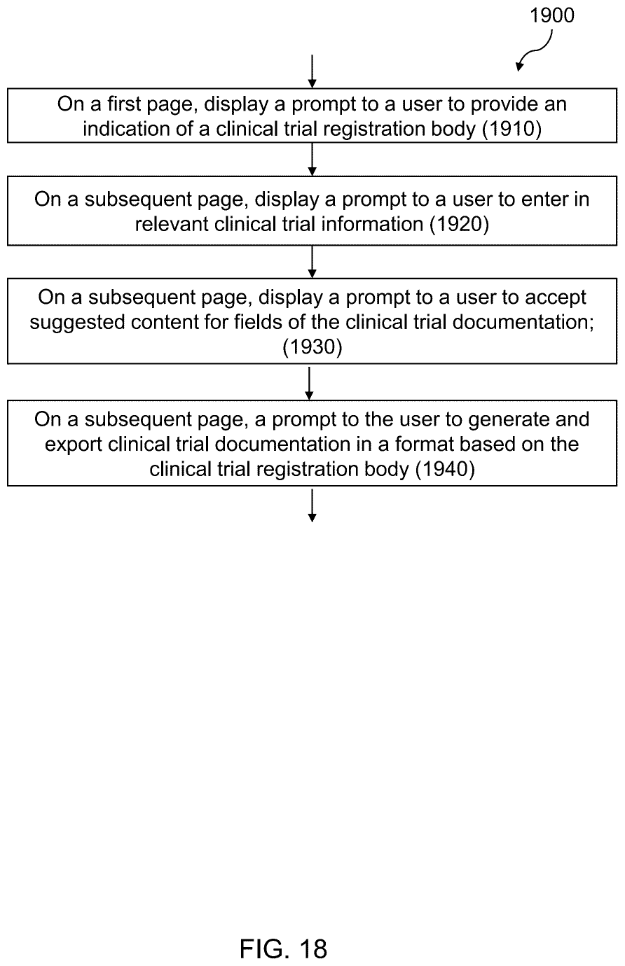

As described above, the computer implemented method may be implemented via a graphical user interface (GUI). FIG. 18 shows a process flow chart of how the graphical user interface may interact with a user to enable operation of the example computer-implemented methods described above. It will be understood that the graphical user interface may be operating on a computing device including a display screen, such as the example computer system described below with reference to FIG. 20. The graphical user interface may be the graphical user interface substantially as shown in FIGS. 4 to 11 and 14 and function in the manner as described above.

Initially, the graphical user interface on a first page, displays a prompt 1910 to a user to provide an indication of a clinical trial registration body. On a subsequent page, the graphical user interface may display a prompt 1920 to a user to enter in relevant clinical trial information. On a subsequent page, the graphical user interface may display a prompt 1930 to a user to accept suggested content for fields of the clinical trial documentation, and then on a subsequent page, the graphical user interface may display a prompt 1940 to the user to generate and export clinical trial documentation in a format based on the clinical trial registration body.

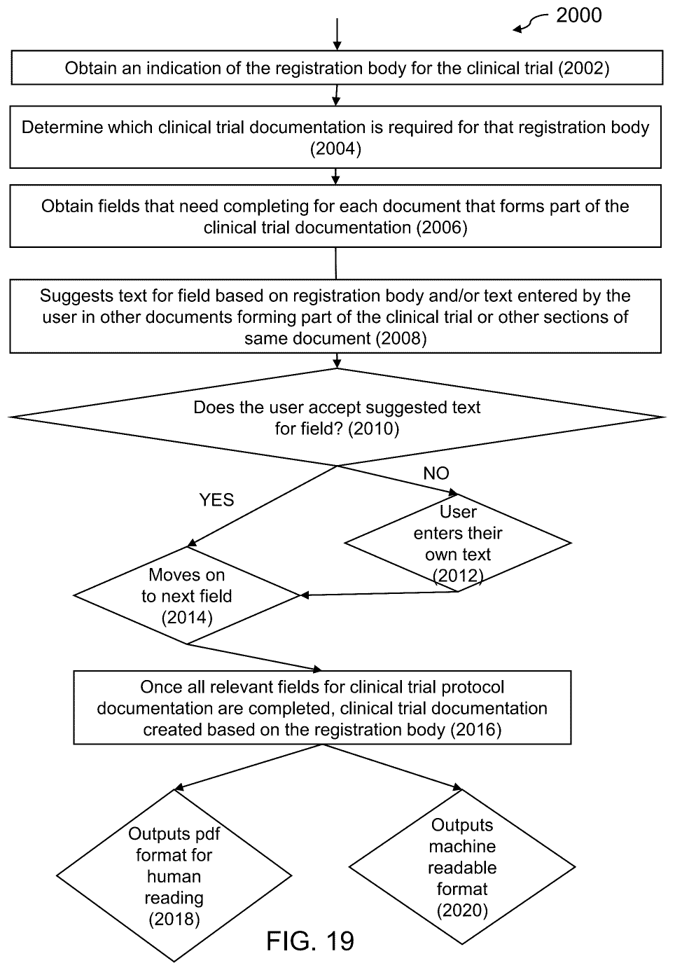

As described above with reference to FIGS. 10 and 11, in some instances the user may be presented with suggested text, for example for entering into a relevant field. The suggested text may be based on text entered by the user in other documents or other sections of the same document. This process is described in more detail below with reference to FIG. 19.

FIG. 19 shows a process flow chart of another example computer-implemented method 2000 for the processing and/or creation of clinical trial protocol documentation. The method begins at step 2002 where it obtains an indication of the registration body for the clinical trial (for example, ISRCTN, EUDRACT or ICTRP). The method then includes determining 2004 which clinical trial documentation is required for that registration body. Determining 2004 which clinical trial documentation is required for that registration body may comprise a lookup in a database of registered protocols for that registration body. For example, registration bodies may have a library or libraries of registered protocols, and the system may be configured to create a local copy of these.

Next, the method includes obtaining 2006 fields that need completing for each document that forms part of the clinical trial documentation. This may include blank labelled fields for the user to enter relevant text/information into. For example, the method may include creating template documents for the user to fill in and complete with relevant information for their clinical trial.

The method then includes suggesting 2008 text for at least one field based. The suggestion may be based on the registration body and/or text entered by the user in other documents forming part of the same clinical trial, or other sections of the same document. However, in other examples the suggestions may be based on other clinical trial documentation. For example, a lookup may be performed for information relevant to each field in other clinical trial documentation, and making a suggestion based on this information in other clinical trial documentation. This other clinical trial documentation may be stored, for example on a local data store such as data store 107 shown in FIG. 1 or data store 207 shown in FIGS. 2 and 3. Additionally, or alternatively, suggesting text for inclusion may comprises suggesting text based on other documents uploaded and/or prepared by the user.

Suggesting text for inclusion may include using a natural language processing model (for example, provided by the ML API 209 described above with reference to FIGS. 2 and 3). The natural language processing model may, for example, be configured to determine section headers for the fields of the clinical trial documentation, and auto-populate at least some of the fields of the clinical trial documentation based on the corresponding determined section header. The section headers may include any of: Title Page, Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling and Recordkeeping, Publication Policy, Project Timetable/Flowchart, References, and Supplements/Appendices.

As described above with reference to FIGS. 10 and 11, the user then chooses 2010 whether to accept the suggested text for the field. If the user accepts 2014, then the suggestion moves 2014 to the next field. If the user declines 2012, the user enters their own text and then the suggestion moves 2014 to the next field. This process repeats until all the fields are completed, and/or no suggestions can be made for the remaining fields.

Once all of the relevant information entered necessary to create all the required clinical trial documentation are entered (as the user will not be entering the relevant information directly into the clinical trial documentation), the clinical trial documentation may be created 2016. The clinical trial documentation may be created based on the registration body—for example, the documentation created for one registration body may differ to that created for another registration body. Creating 2016 completed clinical trial documentation may include creating clinical trial documentation in a format to match ICH GCP guidelines for TMF.

The clinical trial documentation may then be output 2018 in a pdf format for human reading, and also output 2020 in a machine-readable format, for example in xml, json or csv format. For example, the clinical trial documentation may then be converted to a coded format based on the registration body.

Outputting completed clinical trial documentation may additionally or alternatively also include outputting meta data with the clinical trial documentation, wherein the meta data includes at least one of: time stamp, history log, version control.

The meta data may be obtained from different sources depending upon the registry. If the protocol is created using a custom document, then the meta data is extracted from the custom document. If the ID of a registration body is used to get the data, then the meta data is scraped from the respective registration body website. In both cases, the meta is stored in a database such as DynamoDb®. The saved metadata is then visualised to the user as view only mode for the researcher/user, on the system.

It will also be appreciated that a sensitive data file should not be editable directly (making new edits to previous documents without version change) and if it is modified, its history, metadata and numeric version control cannot be maintained without downloading a copy every time before making a change. Accordingly, in some examples sensitive data files can be locked off as uneditable and unmodifiable and whenever changes are requested to that sensitive data file, instead of replacing/modifying the old data file a new version of the data file is created, maintained and locked automatically to a designed user, creating superior automated change management.

FIG. 20 is a block diagram of a computer system 1200 suitable for implementing one or more embodiments of the present disclosure, including for example any of the methods described above with reference to FIGS. 4 to 18. The computer system 1200 may form the function of the protocol controller 105 or anonymization controller 205 described in FIGS. 1 to 3.

The computer system 1200 includes a bus 1212 or other communication mechanism for communicating information data, signals, and information between various components of the computer system 1200. The components include an input/output (I/O) component 1204 that processes a user (i.e., sender, recipient, service provider) action, such as selecting keys from a keypad/keyboard, selecting one or more buttons or links, etc., and sends a corresponding signal to the bus 1212. It may also include a camera for obtaining image data. The I/O component 1204 may also include an output component, such as a display 1202 and a cursor control 1208 (such as a keyboard, keypad, mouse, etc.). The display 1202 may be configured to present a login page for logging into a user account, and is configured to display a user interface such as the user interface described above with reference to FIGS. 5 to 12. An optional audio input/output component 1206 may also be included to allow a user to use voice for inputting information by converting audio signals. The audio I/O component 1206 may allow the user to hear audio. A transceiver or network interface 1220 transmits and receives signals between the computer system 1200 and other devices, such as another user device, a merchant server, or a service provider server via network 1222. In one embodiment, the transmission is wireless, although other transmission mediums and methods may also be suitable. A processor 1214, which can be a micro-controller, digital signal processor (DSP), or other processing component, processes these various signals, such as for display on the computer system 1200 or transmission to other devices via a communication link 1224. The processor 1214 may also control transmission of information, such as cookies or IP addresses, to other devices.

The components of the computer system 1200 also include a system memory component 1210 (e.g., RAM), a static storage component 1216 (e.g., ROM), and/or a disk drive 1218 (e.g., a solid-state drive, a hard drive). The computer system 1200 performs specific operations by the processor 1214 and other components by executing one or more sequences of instructions contained in the system memory component 1210. For example, the processor 1214 can run the systems 100 and 200 described above.

It will also be understood that the modules may be implemented in software or hardware, for example as dedicated circuitry. For example, the modules may be implemented as part of a computer system. The computer system may include a bus or other communication mechanism for communicating information data, signals, and information between various components of the computer system. The components may include an input/output (I/O) component that processes a user (i.e., sender, recipient, service provider) action, such as selecting keys from a keypad/keyboard, selecting one or more buttons or links, etc., and sends a corresponding signal to the bus. The I/O component may also include an output component, such as a display and a cursor control (such as a keyboard, keypad, mouse, etc.). A transceiver or network interface may transmit and receive signals between the computer system and other devices, such as another user device, a merchant server, or a service provider server via a network. In one embodiment, the transmission is wireless, although other transmission mediums and methods may also be suitable. A processor, which can be a micro-controller, digital signal processor (DSP), or other processing component, processes these various signals, such as for display on the computer system or transmission to other devices via a communication link. The processor may also control transmission of information, such as cookies or IP addresses, to other devices.

The components of the computer system may also include a system memory component (e.g., RAM), a static storage component (e.g., ROM), and/or a disk drive (e.g., a solid-state drive, a hard drive). The computer system performs specific operations by the processor and other components by executing one or more sequences of instructions contained in the system memory component.

Logic may be encoded in a computer readable medium, which may refer to any medium that participates in providing instructions to a processor for execution. Such a medium may take many forms, including but not limited to, non-volatile media, volatile media, and transmission media. In various implementations, non-volatile media includes optical or magnetic disks, volatile media includes dynamic memory, such as a system memory component, and transmission media includes coaxial cables, copper wire, and fibre optics. In one embodiment, the logic is encoded in non-transitory computer readable medium. In one example, transmission media may take the form of acoustic or light waves, such as those generated during radio wave, optical, and infrared data communications.

Some common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer is adapted to read.

In various embodiments of the present disclosure, execution of instruction sequences to practice the present disclosure may be performed by a computer system. In various other embodiments of the present disclosure, a plurality of computer systems 1200 coupled by a communication link to a network (e.g., such as a LAN, WLAN, PTSN, and/or various other wired or wireless networks, including telecommunications, mobile, and cellular phone networks) may perform instruction sequences to practice the present disclosure in coordination with one another.

It will also be understood that aspects of the present disclosure may be implemented using hardware, software, or combinations of hardware and software. Also, where applicable, the various hardware components and/or software components set forth herein may be combined into composite components including software, hardware, and/or both without departing from the spirit of the present disclosure. Where applicable, the various hardware components and/or software components set forth herein may be separated into sub-components including software, hardware, or both without departing from the scope of the present disclosure. In addition, where applicable, it is contemplated that software components may be implemented as hardware components and vice-versa.

Software in accordance with the present disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The various features and steps described herein may be implemented as systems including one or more memories storing various information described herein and one or more processors coupled to the one or more memories and a network, wherein the one or more processors are operable to perform steps as described herein, as non-transitory machine-readable medium including a plurality of machine-readable instructions which, when executed by one or more processors, are adapted to cause the one or more processors to perform a method including steps described herein, and methods performed by one or more devices, such as a hardware processor, user device, server, and other devices described herein.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

As utilized herein, terms “component,”, “controller”, “system,” “interface,” “unit” and the like are intended to refer to a computer-related entity, hardware, software (e.g., in execution), and/or firmware. For example, a component can be a processor, a process running on a processor, an object, an executable, a program, a storage device, and/or a computer. By way of illustration, an application running on a server and the server can be a component. One or more components can reside within a process, and a component can be localized on one computer and/or distributed between two or more computers.

Further, these components can execute from various computer readable media having various data structures stored thereon. The components can communicate via local and/or remote processes such as in accordance with a signal having one or more data packets (e.g., data from one component interacting with another component in a local system, distributed system, and/or across a network, e.g., the Internet, a local area network, a wide area network, etc. with other systems via the signal).

As another example, a component can be an apparatus with specific functionality provided by mechanical parts operated by electric or electronic circuitry; the electric or electronic circuitry can be operated by a software application or a firmware application executed by one or more processors; the one or more processors can be internal or external to the apparatus and can execute at least a part of the software or firmware application. As yet another example, a component can be an apparatus that provides specific functionality through electronic components without mechanical parts; the electronic components can include one or more processors therein to execute software and/or firmware that confer(s), at least in part, the functionality of the electronic components. In some cases, a component can emulate an electronic component via a virtual machine, e.g., within a cloud computing system.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

It will be appreciated from the discussion above that the embodiments shown in the figures are merely exemplary, and include features which may be generalised, removed or replaced as described herein and as set out in the claims. In the context of the present disclosure other examples and variations of the apparatus and methods described herein will be apparent to a person of skill in the art.

1. A computer-implemented method for processing and/or creation of clinical trial protocol documentation, the method comprising:
obtaining an indication of a registration body for the clinical trial;
obtaining fields that need completing for each document that forms part of the clinical trial documentation;
auto-populating at least some of the fields of the clinical trial documentation with suggested text; and
outputting completed clinical trial documentation as a machine-readable file, in a format based on the indication of the registration body.

2. The computer-implemented method of claim 1, wherein auto-populating at least some of the fields of the clinical trial documentation comprises using a natural language processing model to determine section headers for the fields of the clinical trial documentation, and auto-populating at least some of the fields of the clinical trial documentation based on a corresponding determined section header.

3. The computer-implemented method of claim 2, wherein the section headers comprise any of: Title Page, Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling and Recordkeeping, Publication Policy, Project Timetable/Flowchart, References, and Supplements/Appendices.

4. The computer-implemented method of claim 1, wherein auto-populating at least some of the fields of the clinical trial documentation comprises auto-populating at least some of the fields of the clinical trial documentation with suggested text based on the registration body and based on other documents uploaded and/or prepared by a user.

5. The computer-implemented method of claim 1, wherein auto-populating at least some of the fields of the clinical trial documentation comprises auto-populating at least some of the fields of the clinical trial documentation with suggested text based on other fields and/or documents completed by a user.

6. A computer-implemented method for processing and/or creation of clinical trial protocol documentation, the method comprising:
obtaining an indication of the registration body for the clinical trial;
based on the obtained indication of a registration body, determining which clinical trial documentation is required for that registration body;
creating template documents for completion by a user based on the determination of which clinical trial documentation is required for that registration body;
suggesting text for inclusion in the template documents based on the registration body and based on text entered by the user in other documents or other sections of the same document; and
outputting completed clinical trial documentation as a coded document, in a format based on the indication of the registration body.

7. The computer-implemented method of claim 6, wherein suggesting text for inclusion comprises suggesting text based on the registration body and/or based on other documents uploaded and/or prepared by the user.

8. The computer-implemented method of claim 7, wherein suggesting text for inclusion comprises suggesting text based on other fields and/or documents completed by the user.

9. The computer-implemented method of claim 6, wherein suggesting text for inclusion comprises using a natural language processing model to determine section headers for the fields of the clinical trial documentation and providing a suggestion based on the relevant section header.

10. The computer-implemented method of claim 9, wherein the section headers comprise any of: Title Page (General Information), Background Information, Objectives/Purpose, Study Design, Selection and Exclusion of Subjects, Treatment of Subjects, Assessment of Efficacy, Assessment of Safety, Adverse Events, Discontinuation of the Study, Statistics, Quality Control and Assurance, Ethics, Data handling and Recordkeeping, Publication Policy, Project Timetable/Flowchart, References, and Supplements/Appendices.

11. The computer-implemented method of claim 6, wherein determining which clinical trial documentation is required for that registration body comprises a lookup in a database of registered protocols for that registration body.

12. The computer-implemented method of claim 6, wherein outputting completed clinical trial documentation comprises outputting clinical trial documentation in a format to match ICH GCP guidelines for TMF.

13. The computer-implemented method of claim 6, wherein outputting completed clinical trial documentation also comprises outputting meta data with the clinical trial documentation, wherein the meta data comprises at least one of: time stamp, history log, version control.

14. The computer-implemented method of claim 6, wherein outputting completed clinical trial documentation also comprises outputting a human-readable set of clinical trial documentation.

15. The computer-implemented method of claim 6, wherein the output completed clinical trial documentation are restricted from editing.

16. The computer-implemented method of claim 6, further comprising obtaining other documents uploaded and/or prepared by the user and converting these to a first machine-readable format, and wherein outputting completed clinical trial documentation comprises converting the completed clinical trial documentation to a second format, wherein the second format is based on the indication of the registration body.

17. The computer-implemented method of claim 6, further comprising obtaining other documents for that registration body, and converting these to a first machine-readable format, and wherein outputting completed clinical trial documentation as a coded document, comprises converting the completed clinical trial documentation to a second format, wherein the second format is based on the indication of the registration body.

18. The computer-implemented method of claim 6 wherein, subsequent to outputting completed clinical trial documentation, the method comprises:
receiving a request by a user to make a change to data in a first document; and
implementing the requested change to all other documents where that same data is used in other documents for that same clinical trial.

19. A computer-implemented method for processing and/or creation of clinical trial protocol documentation, the method comprising:
obtaining completed clinical trial documentation;
receiving a request by a user to make a change to data in a first document; and
implementing the requested change to all other documents for the same clinical trial where that same data is used.

20. A computing device comprising a display screen, the computing device being configured to display on the screen:
a prompt to a user to provide an indication of a clinical trial registration body;
a prompt to a user to enter in relevant clinical trial information;
a prompt to a user to accept suggested content for fields of clinical trial documentation; and
a prompt to the user to generate and export clinical trial documentation in a format based on the clinical trial registration body.
